(19) United States
(12) Patent Application Publication
(10) Pub. No.: US 2003/0119069 A1
(43) Pub. Date: Jun. 26, 2003

(54) Labeling of protein samples

(75) Inventors: Luke V. Schneider, Half Moon Bay, CA (US); Michael P. Hall, San Carlos, CA (US); Robert M. Petesch, Newark, CA (US); Jeffrey N. Peterson, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Target Discovery, Inc., San Carlos, CA

(21) Appl. No.: 10/298,268

(22) Filed: Nov. 15, 2002

Related U.S. Application Data
(63) Continuation of application No. 09/551,937, filed on Apr. 19, 2000, now abandoned.
(60) Provisional application No. 60/130,238, filed on Apr. 20, 1999.

(51) Int. Cl. A61K 51/00; G01N 33/53; C07K 14/00

(52) U.S. Cl. 435/7.1; 530/405; 530/404

(57) ABSTRACT

Provided are methods of labeling multiple proteins in protein mixtures to prepare the samples for identification and analysis, and useful in developing a proteomics database.

### CROSS-REFERENCES TO RELATED APPLICATIONS

This application claims the benefit of U.S. provisional application 60/130,238, filed Apr. 20, 1999. This application is also related to U.S. provisional application 60/075,715 filed Feb. 24, 1998; copending U.S. patent application No. 09/513,486, filed Feb. 25, 2000, entitled “Protein Separation Via Multidimensional Electrophoresis,” and having attorney docket number 020444-000200US; copending U.S. patent application No. 09/513,395, filed Feb. 25, 2000, entitled “Methods for Protein Sequencing,” and having attorney docket number 020444-000300US; copending U.S. application No. 09/513,907, filed Feb. 25, 2000, entitled “Polypeptide Fingerprinting Methods and Bioinformatics Database System,” and having attorney docket number 020444-000100US; copending U.S. patent application No. ______, filed Apr. 19, 2000, entitled “Methods for Conducting Metabolic Analyses”, and having attorney docket number 020444-000400US; and copending PCT application ______, filed Apr. 19, 2000, entitled “Polypeptide Fingerprinting Methods, Metabolic Profiling, and Bioinformatics Database”, and having attorney docket number 020444-000600PC. All of these applications are incorporated by reference in their entirety for all purposes.

### BACKGROUND OF THE INVENTION

The correlation of protein expression levels obtained from healthy and diseased tissue is the basis of proteomics research. Proteins extracted from tissue or cell samples typically must be separated into individual proteins by gel electrophoresis (O'Farrel, P. H., J Biol. Chem., 250:4007 (1975); Hochstrasser, D. F., et al., Anal Biochem., 173:424 (1988); Hühmer, A. F. R., et al., Anal. Chem., 69:29R-57R (1997); Garfin, D. E., Methods in Enzymology, 182:425 (1990)), capillary electrophoresis (Smith, R. D., et al., “Capillary electrophoresis-mass spectrometry,” in: CRC Handbook of Capillary Electrophoresis: A Practical Approach, Chp. 8, pg. 185-206 (CRC Press, Boca Raton, Fla., 1994); Kilár, F., “Isoelectric focusing in capillaries,” in: CRC Handbook of Capillary Electrophoresis: A Practical Approach, Chp. 4, pg. 95-109 (CRC Press, Boca Raton, Fla., 1994); McCormick, R. M., “Capillary zone electrophoresis of peptides,” in: CRC Handbook of Capillary Electrophoresis: A Practical Approach, Chp. 12, pg. 287-323 (CRC Press, Boca Raton, Fla., 1994); Palmieri, R. and Nolan, J. A., “Protein capillary electrophoresis: theoretical and experimental considerations for methods development,” in: CRC Handbook of Capillary Electrophoresis: A Practical Approach, Chp. 13, pg. 325-368 (CRC Press, Boca Raton, Fla., 1994)), or affinity techniques (Nelson, R. W., “The use of affinity-interaction mass spectrometry in proteome analysis,” paper presented at the BC Proteomics conference, Coronado, Calif. (Jun. 11-12, 1998); Young, J., “Ciphergen Biosystems,” paper presented at the CHI Genomics Opportunities conference, San Francisco, Calif. (Feb. 14-15, 1998)), before quantification and comparison of their relative expression levels to those from comparative samples. The most commonly used proteomics method is 2-D gel electrophoresis using staining and imagining techniques to quantify the protein levels present in the gel (Anderson, N. G. and N. L. Anderson, “Twenty years of two-dimensional electrophoresis: Past, present and future,” Electrophoresis, 17:443 (1996)). However, the detection of low abundance proteins (Anderson, L., “Pharmaceutical Proteomics: Targets, mechanisms and function,” paper presented at the BC Proteomics conference, Coronado, Calif. (Jun. 11-12, 1998); McKee, A., “The Yeast Proteome,” paper resented at the BC Proteomics conference, Coronado, Calif. (Jun. 11-12, 1998)) and the reproducibility of protein staining and quantification techniques (Anderson, L., “Pharmaceutical Proteomics: Targets, mechanisms and function,” paper presented at the BC Proteomics conference, Coronado, Calif. (Jun. 11-12, 1998); McKee, A., “The Yeast Proteome,” paper resented at the BC Proteomics conference, Coronado, Calif. (Jun. 11 - 12, 1998); BioRad Molecular Imager FX and PDQuest 2-D analysis software seminar, presented at the IBC Proteomics conference, Coronado, Calif. (Jun. 11-12, 1998); Franzén, F., et al., Electrophoresis, 18:582 (1997)) have proved suspect.

The development of an automated, quantitative, and reproducible system, capable of analyzing protein expression levels directly from native human tissue samples, is expected to significantly impact the time and costs required to generate comparative protein expression data and potentially improve the quality of these data. The development of a labeled 2-D electrophoresis system would allow more rapid accumulation of protein-disease databases and would further speed the identification of new disease targets.

Despite the 2-D gel problems of gel reproducibility, the importance of proteomics research has already been well established. Steiner and coworkers reported the use of proteomics to understand the toxicology of two preclinical drug candidates in rat liver tissues (see, Steiner, “Proteome methods to profile mechanisms of toxicity” paper presented at the IBC Proteomics conference, Coronado, Calif., Jun. 11-12, 1998). Arnott recently reported the identification of proteins whose expression appears to be related to hypertrophy in congestive heart failure, although, it has yet to be determined if any of these proteins are suitable drug targets (see, Arnott, “Protein differential display and mass spectrometry in the study of congestive heart failure” paper presented at the IBC Proteomics conference, Coronado, Calif., Jun. 11-12, 1998). Witzmann et al. report the use of proteomic studies of bovine testis to better understand the toxicology of 1,3,5-trinitrobenzene and 1,3-dinitrobenzene seen in in vitro tissue slices (see, Witzmann, et al., Electrophoresis, 18:642 (1997)). Franzén et al. report the use of proteomic analysis to identify markers for malignant human breast tumors (see, Franzén et al., Electrophoresis, 18:582 (1997)).

Proteomics research also requires that proteins resolved in the separation process also be identified. Clauser et al. have suggested that proteins can only be unambiguously identified through the determination of PSTs that allow reference to the theoretical sequences determined from genomic databases (see, Clauser, et al., Proc. Natl. Acad. Sci. (USA), 92:5072-5076 (1995)). Li et al. appear to have proven this assertion by finding that the reliable identification of individual proteins by MS fingerprinting degenerated as the size of the comparative theoretical peptide mass database increased (see, Li, et al., Electrophoresis 18:391-402 (1997)). Li et al. also reported that they were only able to obtain peptide maps for the highest abundance proteins in the gel because of sensitivity limitations of the MS, even though their matrix assisted laser desorption MALDI methodology was demonstrated to improve the detection sensitivity over previously reported methods. Clearly, rapid and cost effective protein sequencing techniques will improve the speed and lower the cost of proteomics research.

Historically, techniques such as Edman degradation have been extensively used for protein sequencing. See, Stark, in: Methods in Enzymology, 25:103-120 (1972); Niall, in: Methods in Enzymology, 27:942-1011 (1973); Gray, in: Methods in Enzymology, 25:121-137 (1972); Schroeder, in: Methods in Enzymology, 25:138-143 (1972); Creighton, Proteins: Structures and Molecular Principles (W. H. Freeman, NY, 1984); Niederwieser, in: Methods in Enzymology, 25:60-99 (1972); and Thiede, et al. FEBS Lett., 357:65-69 (1995). However, sequencing by collision-induced dissociation mass spectrometry (MS) methods (MS/MS sequencing) has rapidly evolved and has proved to be faster and require less protein than Edman techniques. See, Shevchenko, A., et al., Proc. Natl. Acad. Sci. (USA), 93:14440-14445 (1996); Wilm, et al., Nature, 379:466-469 (1996); Mark, J., “Protein structure and identification with MS/MS,” paper presented at the PE/Sciex Seminar Series, Protein Characterization and Proteomics: Automated high throughput technologies for drug discovery, Foster City, Calif. (March, 1998); and Bieman, Methods in Enzymology, 193:455-479 (1990).

Two basic strategies have been proposed for the MS identification of proteins after their separation from a protein mixture: 1) mass profile fingerprinting (‘MS fingerprinting’, see James, et al., Biochem. Biophys. Res. Commun., 195:58-64 (1993) and Yates, et al., Anal. Biochem. 214:397-408 (1993)) and 2) sequencing of one or more peptide domains by MS/MS (‘MS/MS sequencing’, see Wilm, et al., Nature, 379:466-469 (1996); Chait, et al., Science, 262:89-92 (1993); and Mann, M., paper presented at the IBC Proteomics conference, Boston, Mass. (Nov. 10-11, 1997)). MS fingerprinting is achieved by accurately measuring the masses of several peptides generated by a proteolytic digest of the intact protein and searching a database for a known protein with that peptide mass fingerprint. MS/MS sequencing involves actual determination of one or more PSTs of the protein by generation of sequence-specific fragmentation ions in the quadrapole of an MS/MS instrument.

Despite some progress in analytical methodology, protein identification remains a major bottleneck in field of proteomics. For example, it can require up to 18 hours to generate a protein sequence tag of sufficient length to allow the identification of a single purified protein from its predicted genomic sequence. Shevchenko, A., et al., Proc. Natl. Acad Sci. (USA), 93:14440-14445 (1996). Moreover, although unambiguous protein identification can be attained by generating a protein sequence tag (PST, see Clauser, K. R., et al., Proc. Natl. Acad Sci. (USA), 92:5072-5076 (1995) and Li, G., M., et al., Electrophoresis, 18:391-402 (1997)), limitations on the ionization efficiency of larger peptides and proteins restrict the intrinsic detection sensitivity of MS techniques and inhibit the use of MS for the identification of low abundance proteins. Furthermore, limitations on the mass accuracy of time of flight (TOF) detectors can also constrain the usefulness of presently utilized methods of MS/MS sequencing, requiring that proteins be digested by proteolytic and/or chemolytic means into more manageable peptides (see Ambler, R. P., in: Methods in Enzymology, 25:143-154 (1972) and Gross, E., in: Methods in Enzymol., 11:238-255 (1967) prior to sequencing.

In view of the increasing demand for proteomics data, methods are needed for the separation and sequencing of complex protein samples. Moreover, new protein labeling methods are needed to further facilitate separation and sequencing of proteins in a complex sample. Surprisingly, the present invention provides new methods for the labeling of protein mixtures. The techniques described herein can speed the process of protein identification and correlation of protein expression to disease conditions or development science.

### SUMMARY OF THE INVENTION

The present invention provides a method of labeling a plurality of different proteins in a protein sample, the method comprising contacting the protein sample with a labeling agent comprising a unique ion mass signature component, a quantitative detection component and a reactive functional group to covalently attach a label to at least a portion of the plurality of different proteins. Preferably, the protein sample comprises at least five, more preferably at least 10, more preferably at least 50 and still more preferably at least 100 different proteins. The protein sample used herein is preferably from a biological sample (e.g., cells, tissues, fluids and organs of bacteria, plants, animals, and humans).

The labeling agents used herein have a unique ion mass signature component, a quantitative detection component and a reactive functional group. Preferred quantitative detection components are selected from radioisotopes, fluorescent residues and chromophores. Other detection enhancement components are groups that impart a positively charged or negatively charged ionic species under fragmentation conditions in a mass spectrometer ionization chamber. Suitable groups include quaternary ammonium, quaternary phosphonium and quaternary aryl and alkyl borate groups. Preferred reactive functional groups are selected from functional groups reactive to primary amines and functional groups reactive to carboxylic acids. Suitable amine reactive groups include N-hydroxysuccinimide esters and isothiocyanates. Suitable carboxylic acid reactive groups include primary amines coupled through carbodiimide chemistries and anhydride chemistries. Preferred unique ion mass signature components are those groups that impart a mass to a protein fragment that does not match a residue mass for any of the 20 natural amino acids. Further preferred unique ion mass signature components are those that impart a mass to a protein fragment of from about 100 amu to about 700 amu. Still other preferred unique ion mass signature components are those that incorporate a ratio of stable isotopes into the labeling agent, preferably stable isotopes such as ²H, ¹³C, ¹⁵N and ³⁷Cl. More preferably, the number of stable isotopes incorporated into the label is sufficient to impart a 5 to 20 atomic mass unit difference between the isotopically-enriched and isotopically-depleted forms of the label. Most preferably, the ratio of isotopically-enriched and isotopically-depleted forms of the label are about equimolar.

In other embodiments, the labeling agent can be a mixture of labeling agents comprising two different unique ion mass signature components.

### BRIEF DESCRIPTION OF THE DRAWINGS

FIG. 1 provides examples of “hard” and “soft” positively charged labels suitable for N-terminal sequencing and enhanced fluorescent detection of proteins by methods described herein and in U.S. application Ser. Nos. 09/513,395, 09/513,486 and 09/513,907.

FIG. 2 provides examples of “hard” and “soft” negatively charged labels suitable for N-terminal sequencing and enhanced fluorescent detection of proteins by methods described herein and in U.S. application Ser. Nos. 09/513,395, 09/513,486 and 09/513,907.

FIG. 3 provides examples of compounds suitable for C-terminal labeling through carbodiimide and anhydride attachment chemistries for C-terminal sequencing and enhanced fluorescent detection of proteins by methods described herein and in U.S. application Ser. Nos. 09/513,395, 09/513,486 and 09/513,907.

### DETAILED DESCRIPTION OF THE INVENTION

General

The chemical modification of proteins to facilitate their direct detection is not new, particularly for protein separations conducted in capillary electrophoresis. See, Palmieri, R. and Nolan, J. A., “Protein capillary electrophoresis: theoretical and experimental considerations for methods development,” in: CRC Handbook of Capillary Electrophoresis: A Practical Approach, Chp. 13, pg. 3 25-368 (CRC Press, Boca Raton, Fla., 1994); Pritchett, T., et al., “Quantitation of bioactive peptides in serum by capillary electrophoresis with laser-induced fluorescence immunodetection (CE-LIF-ID), Application Information, A-1791A (Beckman Instruments, Fullerton, Calif. 1995); Jorgenson, J. W. and K. D. Lukacs, J. High Resolut. Chromatogr. Chromatogr. Commun., 4:230 (1981); Bodhe, A. M., et al., Anal. Biochem., 164:39-43 (1987); and Guzman, N. A., et al., J Chromatogr., 608:197-204 (1992). Fluorescamine has been used for the detection of proteins in gels after electrophoresis and in electroblots. See, Vandekerckhoye, J., Eur. J. Biochem., 152:9-19 (1985). However, the direct fluorescent labeling of a plurality of proteins to facilitate their detection after 2-D electrophoretic separation techniques does not appear to have been previously reported.

Rose and Jorgensen (J. Chromatogr., 447:117 (1988)) used o-phthaldialdehyde to derivatize the effluent from a capillary zone electrophoresis separation to enhance post-capillary fluorescence detection. Pritchett et al. (“Quantitation of bioactive peptides in serum by capillary electrophoresis with laser-induced fluorescence immunodetection (CE-LIF-ID), Application Information, A-1791A (Beckman Instruments, Fullerton, Calif. 1995)) demonstrated a competitive fluorescent immunoassay using an antigen (An) labeled with the Cy5™ cyanine dye (at a 1:1 molar ratio) and laser induced fluorescence (LIF) detection after CE separation of the immune reaction product from human serum. They report a detection sensitivity of 10⁻⁹ M for the angiotensin II antigen (An) with the competitive CE immunoassay. Absolute detection sensitivity of the CE-LIF system was not reported, but can be estimated from the reported dilution factor (10) and probable sample loading (10-20 μL) to be about 10⁶ molecules, almost 3 orders-of-magnitude better than the comparable sensitivity of silver staining (assuming a 40 kDa average protein).

The use of chemical derivatization is also the basis of many protein identification techniques (Stark, G. R., Methods in Enzymology, 25:103-120 (1972); Niall, H. D., “Automated Edman degradation: the protein sequenator,” in: Methods in Enzymology, 27:942-1011 (1973); Gray, W. R., Methods in Enzymology, 25:121-137 (1972); Schroeder, W. A., Methods in Enzymology, 25:138-143 (1972); Creighton, T. E., Proteins: Structures and Molecular Principles (W. H. Freeman, NY, 1984); Niederwieser, A., Methods in Enzymology, 25:60-99 (1972)) where the N- or C-terminal amino acid is covalently labeled with a molecule that facilitates its detection in chromatographic analyses conducted after it is enzymatically or chemically cleaved from the protein. Wu and coworkers (Wu, et al., Anal. Biochem., 235:161-174 (1996); Watson, J. T. and J. Wu, Polym. Prepr., 37:3 18 (1996)), Denslow and Nguyen (Denslow, N. D and H. P. Nguyen, in: Techniques in Protein Chemistry VII, Marshak, D. R., ed., pg.. 241-248 (Academic Press, San Diego, Calif., 1996)) and Ming et al. (Ming, D., et al., BioTechniques, 18:808-810 (1995)) have reported methods for the chemical derivatization of cysteine and cystine groups to facilitate identification of the number of such groups and their positions in proteins by MALDI-TOF mass spectrometry (MS). Murphy and Fenselau (Murphy, C. M. and C. Fenselau, Anal. Chem., 67:1644-1645 (1995)) demonstrated the use of methanolysis of the homoserine lactone residues created during cyanogen bromide digestions to add 32 mass units to all internally generated carboxyl terminal peptides. The peptide fragment containing the original C-terminus is converted to a methyl ester, adding only 14 Da to its mass, and thus is distinguishable in the MS. Rose et al. (Rose, K., et al., Biochem. J., 250:253-259 (1988)) demonstrated a similar approach in which tryptic digestion of the protein is conducted in a 1:1 molar ratio of H₂¹⁸O: H₂¹⁶O. Thus, half of the carboxyl termini of the resulting internally generated tryptic fragments would be labeled with ¹⁸O and exhibit a unique 50:50 isotopic split in the resulting mass spectrum. The original carboxyl terminus would remain unlabeled and be easily distinguished.

The present invention resides in a labeling procedure for a protein or mixture of proteins that simultaneously prepares the protein(s) for high precision post-separation detection and subsequent mass spectrometric sequencing and identification, which is preferably nonproteolytic and nonchemolytic. The present method is practiced by labeling the N- or C-terminus of an intact protein or mixture of proteins with a label having a unique ion mass component, a quantitative detection component and a reactive functional group for attaching to the protein or proteins. The resulting labeled protein mixture can then be separated and detected according to methods described in copending application Ser. No. 09/513,486, and the separated labeled proteins can be identified using mass spectrometric methods described in, for example, co-pending application Ser. No. 09/513,395.

Typically, these separation methods involve a conducting a plurality of capillary electrophoretic methods (dimensions), wherein samples containing a plurality of proteins are labeled in a dimension prior to the last electrophoretic separation dimension and labeled protein detection and quantification is conducted at the end of the last electrophoretic dimension. In some embodiments, protein detection and quantitation is accomplished by laser induced fluorescence. In some embodiments, this process results in the detection of 0.01 to 0.001 ng of labeled protein, yielding a 10 to 100-fold more sensitive detection method than current gel staining techniques. In some embodiments, this process results in a 1 to 5% standard deviation in the relative abundance of proteins contained in a sample, yielding a 10-fold more reproducible measure of protein abundance than current gel staining techniques.

The protein identification methods will typically involve fragmenting the intact labeled protein in the ionization zone of a mass spectrometer (e.g., in-source fragmentation) and determining the sequence from the mass ladder of the resulting labeled peptide series. Labeled peptides are differentiated from unlabeled peptides by their unique mass signature in the resulting mass spectrum. In some embodiments, this process is accomplished in less than 1 min for a purified labeled protein, yielding a 500 to 1000-fold more rapid method than current MS/MS protein sequencing techniques.

The labeled proteins are highly fragmented in the ionization zone of the MS, in a manner that is preferably influenced by the presence of the label. Preferred labels lead to increased ionization efficiency and enhanced volatility of the resulting labeled peptide fragment ions, relative to the parent protein, thus improving the overall detection sensitivity. The sequence of the protein or protein sequence tag is preferably constructed from the low molecular weight end of the mass spectrum, providing advantages over prior methods, such as greater absolute mass accuracy and more facile sequencing, including resolution of Q and K residues, from the resulting labeled peptide fragments.

The selection of an appropriate label for this technique requires consideration of several criteria. First, the label is preferably robust enough to survive the fragmentation conditions of the MS. For example, sulfur-containing labels are generally less robust their non-sulfur containing analogs. Second, the label preferably also creates a unique mass/charge (m/z) signature that is distinguishable from any unlabeled peptides generated from internal scissions of the protein backbone. Third, the label will also carry a quantitative detection enhancement component such as an ionizable or permanently ionized group to ensure that fragmentation produces high-abundance ions that include even uncharged N- and C-terminal residues, and/or a chromophoric or fluorophoric group that can be optically detected with high sensitivity.

### DESCRIPTION OF THE EMBODIMENTS

In one aspect, the present invention provides a method of labeling a plurality of different proteins in a protein sample, the method comprising contacting the protein sample with a labeling agent having a unique ion mass signature component, a quantitative detection component and a reactive functional group to covalently attach the label to at least a portion of the plurality of different proteins.

In this aspect of the invention the proteins can be obtained from essentially any source. Preferably, the proteins are at least partially isolated or purified to be free of interfering components. The isolated proteins can be contacted with a labeling moiety, preferably a C-terminus or N-terminus labeling moiety to covalently attach a label to the C- or N-terminus of at least a portion of the proteins to form a mixture of labeled proteins, suitable for further purification and analysis by mass spectrometric fragmentation methods.

Protein Mixtures

Suitable protein samples can be obtained from essentially any biological sample, such as a cell, or a tissue sample derived from a patient. In a preferred embodiment, a sample is obtained from human cells in fluids (e.g., blood, cerebral spinal fluid, and the like) or other tissues such as cells derived from, for example, biopsy or necropsy samples of tumors. Although the sample is typically taken from a human patient, the samples can also be prepared from cells from eukaryotes in general, including plants, vertebrates and invertebrates, and in mammals in particular, such as dogs, cats, sheep, cattle and pigs, and most particularly primates such as humans, chimpanzees, gorillas, macaques, and baboons, and rodents such as mice, rats, and guinea pigs. Microbial cultures can also be used as a source of protein samples.

The cell or tissue sample from which the protein sample is prepared is typically taken from a patient suspected of having, for example, cancer or another disease. Methods of isolating cell and tissue samples are well known to those of skill in the art and include, but are not limited to, aspirations, tissue sections, needle biopsies, and the like. Frequently the sample will be a “clinical sample” which is a sample derived from a using any standard technique known to the skilled artisan. For example, the host cells can be lysed to release the contents of the cytoplasm by French press, homogenization, and/or sonication. The homogenate can then be centrifuged.

For protein samples wherein the polypeptide has formed inclusion bodies in the periplasm, the inclusion bodies can often bind to the inner and/or outer cellular membranes and thus will be found primarily in the pellet material after centrifugation. The pellet material can then be treated with a chaotropic agent such as guanidine or urea to release, break apart, and solubilize the inclusion bodies. Transmembrane and lipophilic proteins can be solubilized from the pellet material after centrifugation through the use of chaotropic agents, surfactants and organic solvents. The polypeptides in their soluble form can then be isolated by immunoprecipitation, acid precipitation (e.g., 5-10% trichloroacetic acid), ammonium sulfate precipitation, solvent precipitation, or other methods known to those of skill in the art. Other methods of protein isolation are described in, for example, Marston et al. Meth. Enz., 182:264-275 (1990).

For protein and polypeptide mixtures wherein inclusion bodies are not formed to a significant degree in the periplasm of the host cell, the polypeptides will be found primarily in the supernatant after centrifugation of the cell homogenate, and the polypeptides can be isolated from the supernatant using methods such as various types of chromatography (immunoaffinity, molecular sieve, and/or ion exchange), and/or high pressure liquid chromatography. In some cases, it may be preferable to use more than one of these methods for complete purification.

Following isolation of protein or polypeptide mixtures as noted, the protein samples will typically be diluted in an appropriate buffer solution or, in some instances, be concentrated. Any of a number of standard aqueous buffer solutions, employing one of a variety of buffers, such as phosphate, tris(hydroxymethyl)aminomethane, or the like, at physiological pH can be used. The protein sample from sources stated above can contain at least five, at least ten, at least 50 or at least 100 or more proteins.

Labeling Agents

A variety of labeling agents are useful in the present invention. Selection of an appropriate labeling agent requires consideration of several criteria, selected from:

i) the mass of the label is preferably unique and preferably shifts the fragment masses to regions of the mass spectrum with low background;

ii) the label preferably contains fixed positive or negative charges to direct remote charge fragmentation at the N- or C-terminus;

iii) the label is preferably robust under the fragmentation conditions and does not undergo unfavorable fragmentation;

iv) the labeling chemistry is preferably efficient under a range of conditions, particularly denaturing conditions, thereby reproducibly and uniformly labeling the N- or C-terminus;

v) the labeled protein preferably remains soluble in the CE and MS buffer systems of choice; and

vi) the label preferably increases the ionization efficiency of the protein, or at least does not suppress it;

vii) the label may contain a mixture of two or more isotopically distinct species to generate a unique mass spectrometric pattern at each labeled fragment position.

In view of the label selection criteria, preferred labeling moieties are those that have a detection enhancement component, an ion mass signature component and a C-terminus or N-terminus reactive functional group. The reactive group can be directly attached to either or both of the other two label components.

In another embodiment, the reactive functional group is separated from one or both of the detection enhancement component and the ion mass signature component by a linker. The linker is preferably designed such that it is chemically stable and inert, and such that it allows efficient separation of the reactive group and at least one of the other two components of the tag. Within a preferred embodiment of the invention, the linker is composed of a hydrocarbon or polyethylene oxide chain or, most preferably, of a hydrocarbon or polyethylene oxide chain linked to an aryl or heteroaryl ring and preferably provides additional separation between the ionizable group and the reactive functional group.

As will be understood by one of ordinary skill in the art, a variety of hydrocarbon chains and modified hydrocarbon chains can be utilized within the present invention. Preferred hydrocarbon chains which are attached to a phenyl ring and are alkylene groups. Particularly preferred linkers range from 2 carbon atoms to about 20 carbon atoms in length. Within a preferred embodiment of the invention, the linker is a phenethyl group.

Ion Mass Signature Component

The ion mass signature component is the portion of the labeling moiety which preferably imparts a unique ion mass signature in mass spectrometric analyses. The sum of the masses of all the constituent atoms of the label is preferably uniquely different than the fragments of all the possible amino acids. As a result, the labeled amino acids and peptides are readily distinguished from unlabeled amino acids and peptides by their ion/mass pattern in the resulting mass spectrum. In a preferred embodiment, the ion mass signature component imparts a mass to a protein fragment produced during mass spectrometric fragmentation that does not match the residue or a- and b-ion mass for N-terminal sequencing, or y-ion mass for C-terminal sequencing for any of the 20 natural amino acids.

As will be understood by one of skill in the art, spurious mass spectral peaks can arise not only from the fragmentation of unlabeled amino acids and peptides but also from impurities in the sample and/or matrix. In order to further increase the uniqueness of the ion mass signature of the label and to be able to identify desired labeled fragment peaks amongst this “noise,” it is preferable to shift the labeled fragments to regions of less spectral noise by optimizing the mass of the label. For example, it is preferred that the label mass generate an ion greater than 100 amu and less than 700 amu. This may be done by increasing the molecular weight of a low molecular weight label or by increasing the number of charges on a high molecular weight label.

An alternative method for providing a more unique mass signature to a labeling moiety is to incorporate stable isotopes in the label (see, for example, Gygi et al., Nature Biotechnol. 17:994-999 (1999)). For example, by incorporating eight deuterium atoms into a labeling moiety and labeling the protein with a 50:50 mixture of the deuterated and nondeuterated label, the resulting singly-charged fragments that include the label are easily identified as equally intense doublets; one at the mass corresponding to the species with the nondeuterated label and the other at the mass corresponding to the species with the deuterated label with a spacing of 8 amu. In a preferred embodiment, the mass difference is from about 1 to about 20 amu at the single charge state. In the most preferred embodiment the mass difference is from about 4 to about 10 amu at the single charge state.

Another method for providing a more unique mass signature to a labeling moiety is to incorporate a mixture of alkyl and/or aryl substitutions onto the label, such that the corresponding set of fragment peaks is easily recognizable in the mass spectrum. For example, the protein can be labeled with a mixture of a label that contains a trimethyl ammonium group and the same label that contains a dimethylethylammonium group in place of the trimethyl ammonium group. This labeling moiety produces two fragment ion peaks for each amino acid in the sequence that differ by 14 amu from each other. It will be apparent to those skilled in the art that many such combinations can be derived.

Detection Enhancement Components

A detection enhancement component, as used herein, refers to a portion of the labeling moiety that facilitates detection and quantitation of the protein fragments by mass spectroscopy, other spectroscopic methods (e.g., UV/Vis, ESR, NMR and the like), or scintillation counting. Accordingly, in one group of embodiments, the detection enhancement component can provide charged (positively or negatively) ionic species under ionization conditions in a mass spectrometer ionization chamber, such that ionization efficiency of the protein is improved. For many of the detection enhancement components, the amount of ionized species present will depend on the medium used to solubilize the protein. Preferred detection enhancement components (i.e., species that can generate a positive or negative charge) can be classified into two categories: 1) components that carry “hard” charge, and 2) components that carry “soft” charge.

Components that carry “hard” charge are arrangements of atoms that are ionized under all conditions, regardless of medium pH. “Hard” positively-charged detection enhancement components include, but are not limited to, tetraalkyl or tetraaryl ammonium groups, tetraalkyl or tetraaryl phosphonium groups, and N-alkylated or N-acylated heterocyclyl and heteroaryl (e.g., pyridinium) groups. “Hard” negatively-charged detection components include, but are not limited to, tetraalkyl or tetraacyl borate groups.

Components that carry “soft” charge are arrangements of atoms that are ionized at a specific pH, respectively (i.e., bases and acids). Within the context of the current invention, “soft” positive charges include those bases with a pKa of greater than 8, preferably greater than 10, and most preferably greater than 12. Within the context of the current invention, “soft” negative charges include those acids with a pKa of less than 4.5, and preferably less than 2, and most preferably less than 1. At the extremes of pKa, the “soft” charges approach classification as “hard” charges. “Soft” positively-charged detection enhancement components include, but are not limited to, 1°, 2°, and 3° alkyl or aryl ammonium groups, substituted and unsubstituted heterocyclyl and heteroaryl (e.g., pyridinium) groups, alkyl or aryl Schiff base or imine groups, and guanidino groups. “Soft” negatively-charged detection enhancement components include, but are not limited to, alkyl or aryl carboxylate groups, alkyl or aryl sulfonate groups, and alkyl or aryl phosphonate or phosphate groups.

For both “hard” and “soft” charged groups, as will be understood by one of ordinary skill in the art, the groups will be accompanied by counterions of opposite charge. For example, within various embodiments, the counterions for positively-charged groups include oxyanions of lower alkyl organic acids (e.g., acetate), halogenated organic acids (e.g., trifluoroacetate), and organosulfonates (e.g., N-morpholinoethane sulfonate). The counterions for negatively-charged groups include, for example, ammonium cations, alkyl or aryl ammonium cations, and alkyl or aryl sulfonium cations.

The detection enhancement component of the label can also be multiply charged or capable of becoming multiply charged. For example, a label with multiple negative charges can incorporate one or more singly charged species (e.g., carboxylate) or it can incorporate one or more multiply charged species (e.g., phosphate). In a representative example of this embodiment of the invention a species bearing multiple carboxylates, such as, for example a polyaminocarboxylate chelating agent (e.g., EDTP, DTPA) is attached to the protein. Methods of attaching polyaminocarboxylates to proteins and other species are well known in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9:108-117 (1998); Song et al., Bioconjugate Chem., 8:249-255 (1997).

In a similar manner, labels having multiple positive charges can be purchased or prepared using methods accessible to those of skill in the art. For example, a labeling moiety bearing two positive charges can be rapidly and easily prepared from a diamine (e.g., ethylenediamine). In a representative synthetic route, the diamine is monoprotected using methods known in the art and the non-protected amine moiety is subsequently dialkylated with a species bearing one or more positive charges (e.g., (2-bromoethyl)trimethylammonium bromide) (Aldrich)). Deprotection using art-recognized methods provides a reactive labeling species bearing at least two positive charges. Many such simple synthetic routes to multiply charged labeling species will be apparent to one of skill in the art. In another embodiment, a mass spectrometer detection enhancement component may consist of a component that enhances the solubility of the protein in volatile nonaqueous solvents.

While charged labels are preferred, components that are neutral but are in close proximity to protein residues that carry “soft” charge (e.g., lysine, histidine, arginine, glutamic acid, or aspartic acid) can be used as detection enhancement components. In this case, the label carries no ionized or ionizable groups, and the detection enhancement is provided the increased volatility of the protein caused by neutralizing ionizable residues and allowing the amount of volatile organic cosolvent to be increased. When such a component carries a unique ion mass it can also serve for generating a protein sequence tag when a nearby protein residue carries charge. Within the context of the present invention, close proximity is defined as within about 4 residues from the labeled terminus of the protein, and more preferably within about 2 residues of the labeled terminus of the protein. Examples include phenylisothiocyanate and N-acetyl groups.

In another group of embodiments, the detection enhancement component is a detectable moiety that can be detected by, for example, spectroscopy (e.g., UV/Vis, fluorescence, electron spin resonance (ESR), nuclear magnetic resonance (NMR) and the like), or scintillation counting (detection of radioactive isotopes), etc. When the protein is detected by UV/Vis, it is generally desirable to attach a chromophoric label to the protein (e.g., phenyl, naphthyl, etc.). Similarly, for detection by fluorescence spectroscopy, a fluorophore is preferably attached to the protein. For ESR, the detectable moiety can be a free radical, such as a moiety including a nitroxide group. When the protein is detected by an NMR method, the detectable moiety can be enriched with an NMR accessible nuclei, such as fluorine, ¹³C, and the like.

In a presently preferred embodiment, the detectable moiety is a fluorophore. Many reactive fluorescent labels are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., Research Organics (Cleveland, Ohio), GIBCO BRL Life Technologies, Inc. (Gaithersburg, Md.), Fluka Chemica- Biochemika Analytika (Fluka Chemie AG, Buchs, Switzerland), and PE-Applied Biosystens (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

There is a great deal of practical guidance available in the literature for selecting an appropriate fluorophore for a particular detectable tag, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing such molecules for covalent attachment via readily available reactive groups that can be added to a molecule.

The diversity and utility of chemistries available for conjugating fluorophores to other molecules is exemplified by the extensive body of literature on preparing nucleic acids derivatized with fluorophores. See, for example, Haugland (supra); Ullman et al., U.S. Pat. No. 3,996,345; Khanna et a., U.S. Pat. No. 4,351,760. Thus, it is well within the abilities of those of skill in the art to choose a suitable fluorophore and to conjugate the fluorophore to a protein or polypeptide.

In addition to fluorophores that are attached directly to a protein, the fluorophores can also be attached by indirect means. In an exemplary embodiment, a ligand molecule (e.g., biotin) is preferably covalently bound to the protein. The ligand then binds to another molecule (e.g., streptavidin), which is either inherently detectable or covalently bound to a signal system, such as a fluorophore described above. In a variation for proteins denatured with binding surfactants (e.g., sodium dodecyl sulfate), such as those separated in the CGE stage of a multidimensional capillary electrophoretic separation, detection can be facilitated with a detection enhancement component that is non-covalently attached to the protein through a detergent-binding fluorophor such as NanoOrange™ and Sypro™ dyes (Molecular Probes, Inc.). These compounds have several desirable qualities, including the following: 1) excellent reproducibility in binding and therefore in protein quantitation, 2) generality of binding independent of protein type, and 3) fluorescent behavior only when the dye is bound to detergent-coated proteins. In this variation, the unique mass signature component may be separately bound to the protein at the N-terminus or C-terminus through covalent means.

Suitable fluorescent compounds (or fluorophores) include fluorescein and its derivatives, rhodamine and its derivatives, dansyl, umbelliferone, etc. Presently preferred fluorophores of use in conjunction with the methods of the invention are the fluoresceins and rhodamine dyes. Many suitable forms of these compounds are widely available commercially with substituents on their phenyl moieties, which can be used as the bonding functionality for attachment of the fluorophore to a protein. Another group of preferred fluorescent compounds are the naphthylamines, having an amino group in the alpha or beta position. Included among such naphthylamino compounds are 1-dimethylaminonaphthyl-5-sulfonate, 1 -anilino-8-naphthalene sulfonate and 2-p-toluidinyl-6-naphthalene sulfonate. Other suitable fluorophores include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, pyrenes, and the like.

Useful fluorescent detectable moieties can be made to fluoresce by exciting them in any manner known in the art, including, for example, with light or electrochemical energy (see, for example, Kulmala et al, Analytica Chimica Acta 386:1 (1999)). Means of detecting fluorescent labels are well known to those of skill in the art. Thus, for example, fluorescent labels can be detected by exciting the fluorophore with the appropriate wavelength of light and detecting the resulting fluorescence. The fluorescence can be detected visually, by means of photographic film, by the use of electronic detectors such as charge coupled devices (CCDs) or photomultipliers and the like.

The fewer the processing steps between any separation technique and MS sequencing method, the faster that proteins can be identified, and the lower the cost of proteomic research. Typical electrophoresis buffers (e.g., Hochstrasser et al. Anal Biochem., 173:424 (1988). and O'Farrel, J Biol. Chem., 250:4007 (1975)) contain components (e.g., tris(hydroxymethyl)aminomethane buffers and sodium dodecyl sulfate, that supress the ionization of proteins in the mass spectrometer. These components may be replaced with other more volatile components (e.g., morpholinoalkylsulfonate buffers and ephemeral surfactants) that do not suppress ionization in the MS. In another embodiment, the samples are diluted with ammonium bicarbonate or ammonium acetate buffer to provide a volatile proton source for the mass spectrometer. Wilm, M. et al., Anal. Chem., 68:1-8 (1996). In another embodiment, a buffer exchange is conducted by chromatographic or tangential flow dialysis as the sample is transported from the outlet of the separation process to the inlet of the MS.

Reactive Groups

A third component of the labeling moiety is a functional group which is reactive with the N-terminus amino group, the C-terminus amino group or another constituent of the N- or C-terminus amino acid.

The reactive functional group can be located at any position on the labeling agent. For example, the reactive group can be located on an aryl nucleus or on a chain, such as an alkyl chain, attached to an aryl nucleus. When the reactive group is attached to an alkyl, or substituted alkyl chain tethered to an aryl nucleus, the reactive group is preferably located at a terminal position of an alkyl chain. Reactive groups and classes of reactions useful in practicing the present invention are generally those that are well known in the art of bioconjugate chemistry. Currently favored classes of reactions are those which proceed under relatively mild conditions in an aqueous or mixed aqueous/organic solvent milieu.

Particularly preferred chemistries that target the primary amino groups in proteins (including the N-terminus) include, for example: aryl fluorides (see, Sanger, F., Biochem. J., 39:507 (1945); Creighton, T. E., Proteins: Structures and Molecular Principles (W. H. Freeman, NY, 1984); Niederwieser, A., in: Methods in Enzymology, 25:60-99 (1972); and Hirs, C. H. W., et al., Arch. Biochem. Biophys., 111:209-222 (1965), sulfonyl chlorides (Gray, W. R., in: Methods in Enzymology, 25:121-137 (1972)), cyanates (Stark, G. R., in: Methods in Enzymology, 25:103-120 (1972)), isothiocyanates (Niall, H. D., in: Methods in Enzymology, 27:942-1011 (1973)), imidoesters (Galella, G., et al., Can. J. Biochem. 60:71-80 (1982)), N-hydroxysuccinimidyl esters (Lomant, A. J., et al., J. Mol. Biol., 104:243-261 (1976)), O-acylisoureas (Lomant, A. J., et al., J. Mol. Biol., 104:243-261 (1976)), chlorocarbonates and carbonylazides (Solomons, T. W. G, Organic Chemistry (John Wiley & Sons, NY, 1976), aldehydes (Novotny et al., Anal. Chem., 63:408 (1991) and Novotny et al., J. Chromatography, 499:579 (1990)), and alkylhalides and activated alkenes (Wagner, D. S., et al., Biol Mass Spectrometry, 20:419-425 (1991)).

Preferred examples of chemical constituents that react with the carboxyl groups of proteins are benzyl halides (Solomons, T. W. G, Organic Chemistry (John Wiley & Sons, NY, 1976); Merrifield, B., Science, 232:341-347 (1986); and Horton, H. R., et al., Methods in Enzymology, 25:468 (1972)), carbodiimide (Yamada, H., et al., Biochem., 20:4836-4842)), particularly if stabilized using N-hydroxysuccinimide (see, Grabarek, Z., et al., Anal. Biochem. 185:131 -135 (1990)), and anhydrides (see, Werner, et al., “A New Simple Preparation Device for Protein/Peptide Sequencing,” poster presentation at the Ninth Symposium of the Protein Society). The carbodiimide/N-hydroxysuccinimide approach is expected to label carboxyl-containing amino acid residues (e.g., aspartate and glutamate) along with that of the C-terminus. The anhydride approach, however, can uniquely be used to discriminate between the carboxyl residues and C-terminal carboxyl group of the protein. These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

The reactive functional groups can be chosen such that they do not participate in, or interfere with, the reactions necessary to assemble the labeling agent. Alternatively, a reactive functional group can be protected from participating in the reaction by the presence of a protecting group. One of skill in the art will understand how to protect a particular functional group such that it does not interfere with a selected set of reaction conditions. For examples of useful protecting groups, see, for example, Greene et al., PROTECTIVE GROUPS IN ORGANIC SYNTHESIS, John Wiley & Sons, New York, 1991.

A variety of functional groups are described below with references to conditions for appropriate attachment. One of skill in the art will understand that each of the groups noted will be further modified to incorporate a unique ion mass signature component and a quantitative detection component according to established chemical protocols.

In slightly alkaline (pH 8-9) solutions, Sanger's reagent (1-fluoro-2,4-dinitrobenzene) will undergo nucleophilic attack by primary amines to form a stable secondary aryl amine. (Solomons, T. W. G, Organic Chemistry (John Wiley & Sons, NY, 1976)) Sanger's reagent also reacts with the E-amino group of lysine residues, as well as histidine and tyrosine residues at pH 10 and 40° C. (albeit after a well defined lag phase). (Hirs, C. H. W., Arch. Biochem. Biophys., 111:209-222 (1965)) Reductive amination (Solomons, T. W. G, Organic Chemistry (John Wiley & Sons, N.Y., 1976)) can also be used to increase the degree of substitution of an amine using aldehydes and ketones, (Novotny et al., Anal. Chem., 63:408 (1991); Novotny et al., J. Chromatography, 499:579 (1990)) but is of reduced efficiency in aqueous solutions.

Dansyl chloride undergoes a similar nucleophilic attack by the amines in proteins at alkaline pH, producing an aromatic sulfonamide (Gray, W. R., Methods in Enzymology, 25:121-137 (1972)) However, sulfonyl chlorides, depending on the pH, can also react with secondary amines. (Solomons, T. W. G, Organic Chemistry (John Wiley & Sons, NY, 1976)) The aromatic constituent enables fluorescence detection of the reaction product. Dansyl chloride also reacts with the ε-amino group of lysine. (Gray, W. R., Methods in Enzymology, 25:121-137 (1972))

Potassium cyanate can also be used for labeling the amino groups of proteins at alkaline pH. See, Stark, et al., (Stark, G. R., Methods in Enzymology, 25:103-120 (1972)). Similarly, phenylisothiocyanate (Niall, H. D., Methods in Enzymology, 27:942-1011(1973)) has been used to label the amino groups of proteins at alkaline pH. Cyanate forms an N-terminal amide and isothiocyanate forms a thiamide. Isothiocyanates are also commonly used for the attachment of fluorescent labels to proteins. (Hermanson, G., Bioconjugate Techniques (Academic Press, 1995); Haugland, R. P., Handbook of Fluorescent Probes and Research Chemicals (Molecular Probes, Eugene, Oreg., 1996))

Imidoesters are widely used for protein crosslinking (Hermanson, G., Bioconjugate Techniques (Academic Press, 1995); Hartman, F. C. and F. Wold, Biochem., 6:2439-2448 (1967)) and the conjugation of fluorophores or other moieties to proteins. (Haugland, R. P., Handbook of Fluorescent Probes and Research Chemicals (Molecular Probes, Eugene, Oreg., 1996)) Nucleophilic attack of the imidoester by primary amines on the protein at alkaline pH (8-9) result in the formation of an amidine bond. (Browne, D. T. and S. B. H. Kent, Biochem. Biophys. Res. Commun., 67:126-132 (1975)) N-Hydroxylsuccinimidyl (NHS) esters are also commonly used for protein crosslinking and the attachment of labels to proteins through an amide bond. (Hermanson, G., Bioconjugate Techniques (Academic Press, 1995); Haugland, R. P., Handbook of Fluorescent Probes and Research Chemicals (Molecular Probes, Eugene, Oreg., 1996)) NHS esters react with excellent specificity for the primary ε-amino groups of lysine (Cuatrecasas, P. and I. Parikh, Biochem., 11:2291-2299 (1972)) and the α-amino group of the N-terminus of proteins, (Hermanson, G., Bioconjugate Techniques (Academic Press, 1995)) leaving other amine-containing residues intact. The rate of imidylester hydrolysis is controlled by the pH. (Hermanson, G., Bioconjugate Techniques (Academic Press, 1995))

Benzylchlorocarbonate, benzyl chloroformate, and t-butoxycarbonylazide are commonly used to block reactive amine groups in polypeptide synthesis reactions. (Solomons, T. W. G, Organic Chemistry (John Wiley & Sons, NY, 1976); Merrifield, B., Science, 232:341-347 (1986).) Both are highly efficient derivatizing agents under alkaline conditions at room temperature. Both reagents yield similar acid labile amidoesters.

The α-amino group of the N-terminus of polypeptides can be preferentially labeled over the ε-amino groups of lysine by pH control. This is possible due to the difference in pKₐ of the two groups. In model peptides, it has been shown that the α-amino groups react about 100 times faster than ε-amino groups in nucleophilic addition reactions with cyanate (Wetzel et al., Bioconjugate Chem., 1:114-122 (1990)). Furthermore, the added resonance stabilization and increased s-character of the amine lone pairs in arginine and histidine make these residues less reactive than the α-amino group of the N-terminus and these residues are not expected to be significantly labeled under the normal reaction conditions.

Merrifield (Merrifield, B., Science, 232:341-347 (1986)) capitalized on the nucleophilic substitution of benzyl chloride by carboxylate ions (Horton, H. R. and D. E. Koshland, Jr., Methods in Enzymology, 25:468 (1972)) in his classic solid phase peptide synthesis method. The benzyl halide reaction forms a benzyl ester.

Carbodiimides react with carboxyl groups to form an O-acylisourea intermediate that is highly unstable in aqueous solution but can be stabilized through the addition of N-hydroxysuccinimide resulting in the formation of an acid stable intermediate that can be made to react with primary amines at alkaline conditions, producing an amide. (Grabarek, Z. and J. Gergely, Anal. Biochem. 185:131-135 (1990)) The carboxyl terminus, glutamate and aspartate residues are all targets for carbodiimides in proteins at acidic pH (4.5-5). (Hermanson, G., Bioconjugate Techniques (Academic Press, 1995)) Carbodiimide chemistry could be useful for labeling the C-terminus of protein if an excess of primary amines is added to the protein solution to inhibit crosslinking reactions, or in a two-step process involving the use of an amine containing fluorescent molecule through the N-hydroxysuccinimide intermediate.

In the presence of bases (e.g., 2,6-lutidine), acetic anhydride [Dupont, et al., PE Biosystems, Inc. Application Note, http://www.pbio.com] reacts with carboxy groups on proteins either in free solution or immobilized to a solid support (e.g., polyvinylidene fluoride) to form mixed anhydrides.. The resulting C-terminal α-amino anhydride is able to cyclize to form an oxazolone intermediate. The mixed anhydrides formed from glutamate and aspartate residues fail to cyclize. The C-terminal oxazolone is resistant to subsequent nucleophilic addition under basic conditions, while the other anhydrides are not. Therefore, the carboxyl residues can be selectively protected before the C-terminus. The C-terminus can then be labeled by lowering the pH with the addition of acid (e.g., TFA) and subsequently adding primary amines or other nucleophiles carrying suitable detection enhancement and unique mass signature components. However, this approach fails when the C-terminal residue is a proline.

Table 1 provides a non-limiting list of a number of labeling moieties useful in the labels of the present invention.

TABLE 1

| | | Linkage |
|---|---|---|
| Label | Source | Formed |
| Amine Labeling | | |
| 2,4,6-trinitrobenzenesulfonic acid | Aldrich | Aryl amine |
| Lissamine ™rhodamine B sulfonyl | Molecular | Sulfonamide |
| chloride | Probes | |
| 2′,7′-dichlorofluoroscein-5- | Molecular | Thiourca |
| isothiocyanate | Probes | |
| 4,4-difluoro-5,7-dimethyl-4-bora- | Molecular | Amide |
| 3a,4a-diaza-s-indacene-3-propionic | Probes | |
| acid, sulfosuccinimidyl ester | | |
| Naphthalene-2,3 -dicarboxylaldehyde | Molecular | Isoindole |
| | Probes | |
| Carboxyl Labeling | | |
| 5-(bromomethyl)fluorescein | Molecular | Ester |
| | Probes | |
| N-cyclohexyl-N′-(4-(dimethylamino) | Molecular | N-Acylurea |
| naphthyl)carbodiimide | Probes | |
| 1-ethyl-3-(3-dimethylaminopropyl)- | Pierce | Amide |
| carbodiimide hydrochloride with N- | Aldrich | |
| hydroxysuccinimide and 5- | Molecular | |
| aminofluorescein | Probes | |

One of skill in the art will understand that labeling techniques are readily available for a number of the labeling moieties. An example of an N-terminus labeling group (dansyl chloride) and a C-terminus labeling group (carbodiimide) are provided as illustrative of the invention, with references to a more complete description of their use. The focus on these two labeling moieties is for clarity of illustration and does not limit the scope of the invention.

Dansyl chloride undergoes a nucleophilic attack by the amines in proteins at alkaline pH, producing an aromatic sulfonamide. Sulfonyl chlorides, however, depending on the pH, can also react with secondary amines. The aromatic constituent enables spectroscopic (e.g., fluorescence) detection of the reaction product. Dansyl chloride also reacts with the ε-amino group of lysine. The pKₐ differences between α- and ε-amines can be exploited to modify α-amino groups preferentially.

Carbodiimides react with carboxyl groups to form an O-acylisourea intermediate that is highly unstable in aqueous solution but can be stabilized through the addition of N-hydroxysuccinimide resulting in the formation of an acid stable intermediate that can be made to react with primary amines, producing an amide. The carboxyl terminus, glutamate and aspartate residues are all targets for carbodiimides in proteins at acidic pH (4.5-5). Carbodiimide chemistry is useful for labeling the C-terminus of proteins. When carbodiimide chemistry is utilized, it is generally preferred that an excess of amine is added to the protein solution to inhibit crosslinking reactions. In another exemplary embodiment, a protein amine is labeled in a two-step process; an amine-containing fluorescent molecule is tethered to the protein through an N-hydroxysuccinimide intermediate of the protein or of a spacer arm attached to the protein.

Synthesis

Once the reactive group, optional linker, unique ion mass signature component and quantitative detection component have been selected, the final compound can synthesized by one of ordinary skill in the art utilizing standard organic chemistry reactions. A preferred compound for use within the present invention is PETMA-PITC, or an analogous agent. This compound retains the excellent characteristics of phenylisothiocyanate in the coupling. Furthermore, the compound performs well as a label in analytical methods because the electron structure of the phenyl ring is sufficiently separated from the quaternary ammonium group by the ethyl linker, thus allowing the isothiocyanate to react undisturbed by the quaternary ammonium group. Preparation of PETMA-PITC, C5 PETMA-PITC and PITC-311 are described in Aebersold et al., U.S. Pat. No. 5,534,440, issued Jul. 9, 1996.

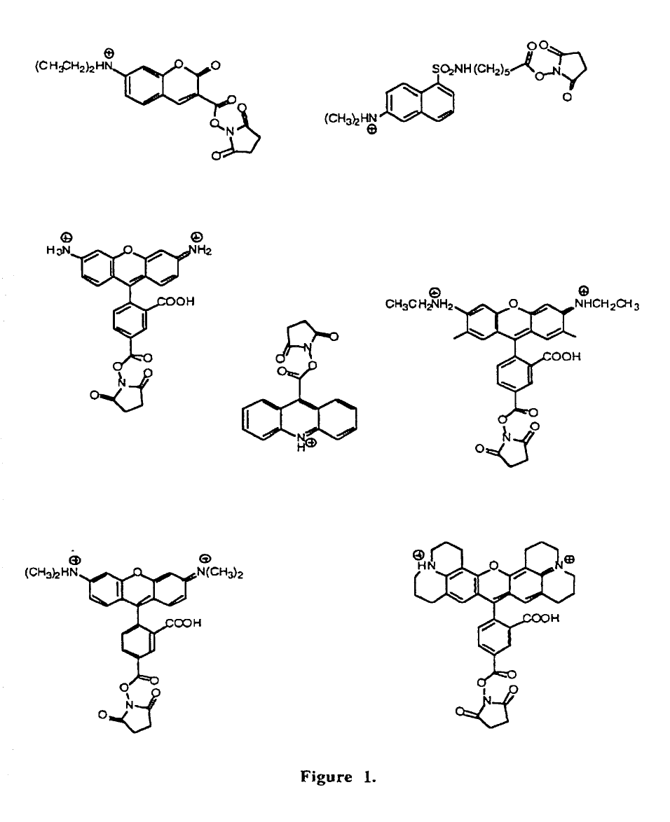

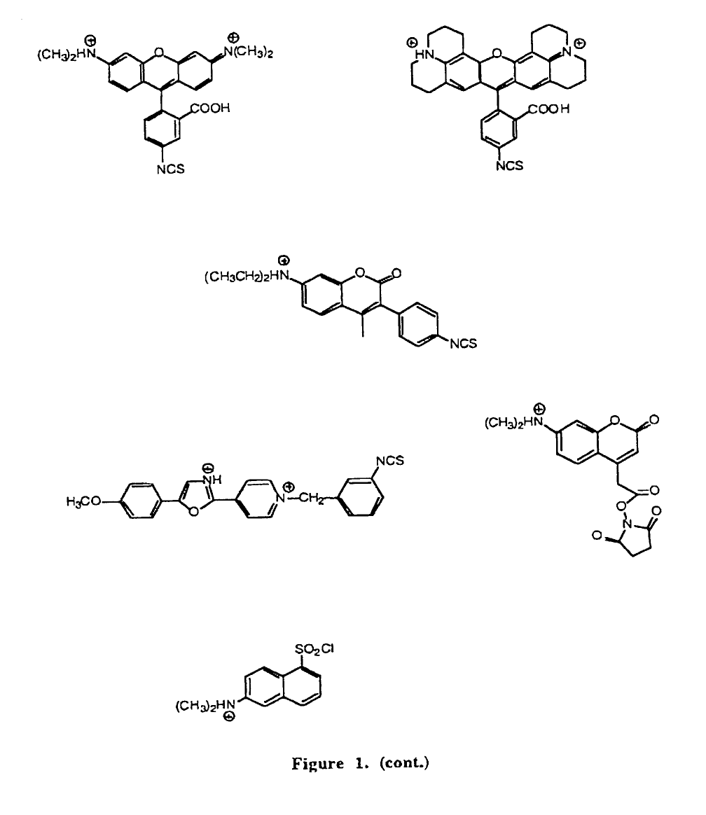

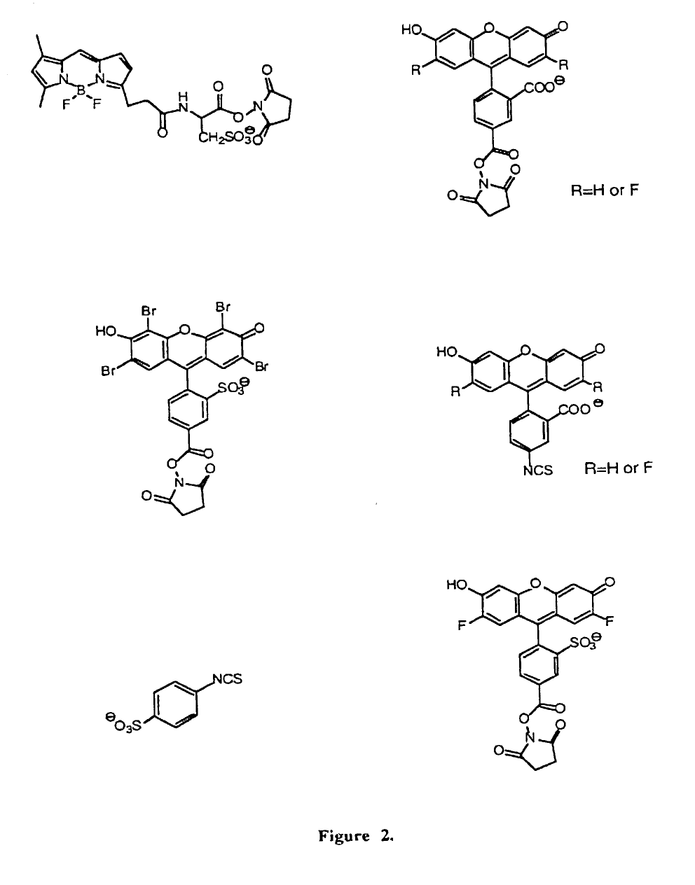

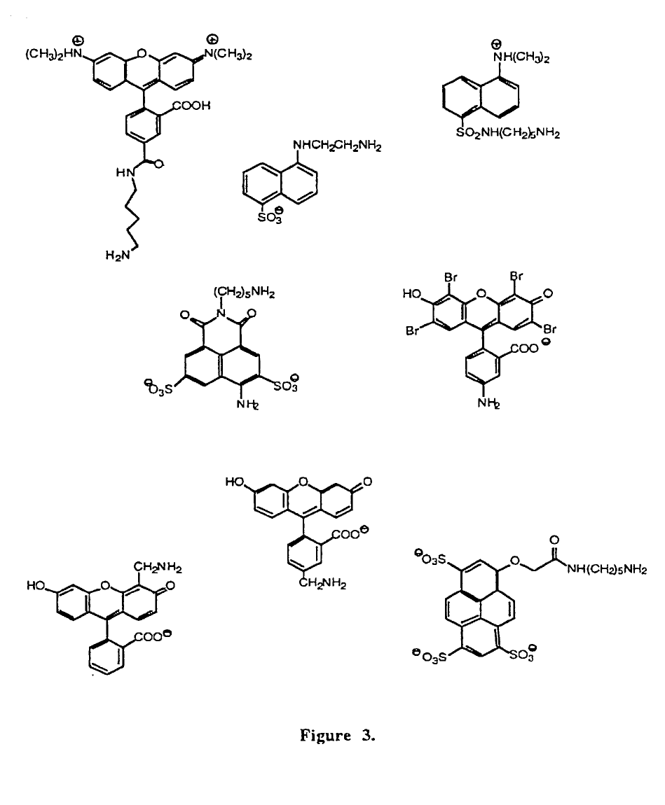

Other suitable commercially available labels that satisfy the labeling agent criteria set forth above, include sulfophenyl isothiocyanate, N-aminopropyl pyridine (attached to the C-terminus through carbodiimide chemistry), and the species shown in FIGS. 1-3. FIGS. 1 and 2 show examples of cationic and anionic fluorescent N-terminal labels bearing NHS-ester, isothiocyanate and sulfonyl chloride reactive groups. Examples include both “hard” and “soft” positive charges (e.g., “hard” charge represented by diallkyl immonium and “soft” charge represented by anilinium) (FIG. 1) and “soft” negative charges (i.e., sulfonate and carboxylate) (FIG. 2). FIG. 3 shows examples of both cationic and anionic fluorescent labels bearing primary amino groups that can be used for C-terminal labeling via the carbodiimide or anhydride approaches. All of the labels in FIGS. 1-3 have a MW≧200; thus, the smallest singly-charged fragment mass of an amino acid would be ˜229 which is the sum of the mass of the a-ion of glycine (29) and the label weight (200). Since the largest fragment of an amino acid (i.e., the y-ion of tryptophan) has a mass of ˜205, all the labels shown would produce initial fragments of interest with masses that are higher, and thus uniquely different, than those of the naturally-occurring amino acids. In a preferred embodiment, the labels shown with higher molecular weights (e.g., the fluorescein derivatives MW>500) would shift the fragment masses to at least 500 or more, which avoids the low m/z range of the MS which tends to be populated with spurious contaminant and matrix peaks.

Labeling Procedure

With the selection of a suitable labeling agent, conditions for attaching the label to the protein should ensure that the N- or C-terminus of the proteins is uniformly labeled and that the labeled protein remains soluble in appropriate buffer systems. Typically, labeling will be carried out under denaturing conditions (e.g., surfactants or 8M urea). Surfactants and urea both suppress MS ionization and methods that provide rapid clean up and transfer of the labeled protein sample to a suitable MS buffer should also be employed.

As noted, some salts (e.g., TRIS and SDS) and urea present in electrophoresis buffers can suppress ionization of the labeled proteins and can generate small mass/charge ions that potentially confuse sequence analysis. Accordingly, spin dialysis procedures can be employed to rapidly exchange buffer systems prior to MS analysis. Alternatively, desalting columns (e.g., the ZipTip™ tip sold by Millipore) can be used for sample clean up and buffer exchange. Desalted samples can be resuspended in 0.1 M ammonium bicarbonate as described by Wilm and Mann (see, Wilm, et al., ibid.) with minimal addition of methanol, or in 0.01M ammonium acetate buffer (with 0.1% formic acid) with minimal addition of acetonitrile as described by Mark (see “Protein structure and identification with MS/MS,” paper presented at the PE/Sciex Seminar Series, Protein Characterization and Proteomics: Automated high throughput technologies for drug discovery, Foster City, Calif. (March, 1998)).

The coupling rates of the compound may be tested to ensure that the compound is suitable for sequencing polypeptides. In general, the faster the coupling rate the more preferred the compound. Coupling rates of between 2 and 10 minutes at 50° C. to 70° C. are particularly preferred. Similarly, fast reaction rates are also preferred, because exposure to the reaction mixture over an extended period of time might hydrolyze the peptide bonds, or lead to inefficient and irreproducible side reactions with the polypeptide residues, which could complicate mass spectral deconvolution.

In another preferred embodiment, one or more of the components of a protein mixture is reversibly attached to a solid support prior to the label being attached to a polypeptide. Various materials may be used as solid supports, including, for example, numerous resins, membranes or papers. These supports may additionally be derivatized to incorporate a cleavable functionality. A number of cleavable groups that may be used for this purpose include disulfides (—S—S—), glycol (—CH[OH]—CH[OH]—), azo (—N═N—), sulfone (—S[═O]—), and ester (—COO—) linkages (see, Tae, Methods in Enzymology, 91:580 (1983)). Supports which are particularly preferred include membranes such as Sequelon™ (Milligen/Biosearch, Burlington, Mass.). Representative materials for the construction of these supports include, among others, polystyrene, porous glass, polyvinylidinefluoride and polyacrylamide. In particular, polystyrene supports include, among others: (1) a (2-aminoethyl) aminomethyl polystyrene (see, Laursen, J. Am. Chem. Soc. 88:5344 (1966)); (2) a polystyrene similar to number (1) with an aryl amino group (see, Laursen, Eur. J. Biochem. 20:89 (1971)); (3) amino polystyrene (see, Laursen et al., FEBS Lett. 21:67 (1972)); and (4)triethylenetetramine polystyrene (see, Horn et al., FEBS Lett. 36:285 (197,)). Porous glass supports include: (1) 3-aminopropyl glass (see, Wachter et al., FEBS Lett. 35:97 (1973)); and (2)N-(2-aminoethyl)-3-aminopropyl glass (see, Bridgen, FEBS Lett. 50:159 (1975)). Reaction of these derivatized porous glass supports with p-phenylene diisothiocyanate leads to activated isothiocyanato glasses (see, Wachter et al., supra). Polyacrylamide-based supports are also useful, including a cross-linked β-alanylhexamethylenediamine polydimethylacrylamide (see, Atherton et al., FEBS Lett. 64:173 (1976)), and an N-aminoethyl polyacrylamide (see, Cavadore et al., FEBS Lett. 66:155 (1976)).

One of ordinary skill in the art will readily utilize appropriate chemistry to couple the polypeptide or protein to the solid supports described above (see, generally Machleidt and Wachter, Methods in Enzymology: [29] New Supports in Solid-Phase Sequencing 263-277 (1974). Preferred supports and coupling methods include the use of aminophenyl glass fiber paper with EDC coupling (see, Aebersold et al., Anal. Biochem. 187:56-65 (1990)); DITC glass filters (see, Aebersold et al., Biochem. 27:6860-6867 (1988) and the membrane polyvinylidinefluoride (PVDF) (Immobilon P™, Milligen/Biosearch, Burlington, Mass.), along with SequeNet™ chemistry (see, Pappin et al., CURRENT RESEARCH IN PROTEIN CHEMISTRY, Villafraica J. (ed.), pp. 191-202, Academic Press, San Diego, 1990)).

In the practice of the present invention, attachment of the polypeptide or protein to the solid support may occur by either covalent or non-covalent interaction between the polypeptide or protein and the solid support. For non-covalent attachment of the polypeptide to the solid support, the solid support is chosen such that the polypeptide attaches to the solid support by non-covalent interactions. For example, a glass fiber solid support may be coated with polybrene, a polymeric quaternary ammonium salt (see, Tarr et al., Anal. Biochem., 84:622 (1978)), to provide a solid support surface which will non-covalently attach the polypeptide. Other suitable adsorptive solid phases are commercially available. For example, polypeptides in solution may be immobilized on synthetic polymers such as polyvinylidine difluoride (PVDF, Immobilon, Millipore Corp., Bedford, Mass.) or PVDF coated with a cationic surface (Immobilon CD, Millipore Corp., Bedford, Mass.). These supports may be used with or without polybrene. Alternatively, polypeptide samples can be prepared for sequencing by extraction of the polypeptide directly from polyacrylamide by a process called electroblotting. The electroblotting process eliminates the isolation of polypeptide from other peptides which may be present in solution. Suitable electroblotting membranes include Immobilon and Immobilon CD (Millipore Corp., Bedford, Mass.).

More recently, automated methods have been developed that allow chemistries to be performed on polypeptides immobilized on solid supports by non-covalent, hydrophobic interaction. In this approach, the samples in aqueous buffers, which may contain salts and denaturants, are pressure-loaded onto columns containing a solid support. The bound polypeptide is then pressure-rinsed to remove interfering components, leaving the bound polypeptide ready for labeling (see, Hewlett-Packard Product Brochure 23-5091-5168E (Nov., 1992) and Horn, U.S. Pat. No. 5,918,273 (Jun. 29, 1999).

The bound polypeptide or protein is reacted under conditions and for a time sufficient for coupling to occur between the terminal amino acids of the polypeptide and the labeling moiety. The physical properties of the support may be selected to optimize the reaction conditions for a specific labeling moiety. For example, the strongly polar nature of the PETMA-PITC dictates covalent attachment of the polypeptide. Preferably, coupling with the amino groups of the polypeptide occurs under basic conditions, for example, in the presence of an organic base such as trimethylamine, or N-ethylmorpholine. In a preferred embodiment, the label is allowed to react with the bound peptide in the presence of 5% N-ethylmorpholine in methanol:water (75:25 v/v). Because of the mode of attachment, excess of reagent, coupling base and reaction by-products can be removed by very polar washing solvents prior to removal and sequencing of the labeled polypeptide by mass spectrometry. Various reagents are suitable as washing solvents, including, for example, methanol, water, mixtures of methanol and water, or acetone.

Less polar reagents, such as PITC-311, may be reacted with polypeptides attached to a sold support preferably by hydrophobic, non-covalent interactions. In this case, less polar washes are preferred, such as heptane, ethylacetate, and chloroform. Following the washing cycle, the labeled polypeptide is dissociated from the solid support by elution with solvent containing 50% to 80% of aqueous methanol or acetonitrile.

When the labeling reaction is conducted entirely in solution phase, the reaction mixture is preferably submitted to a purification cycle, such as dialysis, gel permeation chromatography, and the like.

Still other conditions for labeling proteins can be found in, for example, Means et al., CHEMICAL MODIFICATION OF PROTEINS, Holden-Day, San Francisco, 1971; Feeney et al., MODIFICATION OF PROTEINS: FOOD, NUTRITIONAL AND PHARMACOLOGICAL ASPECTS, Advances in Chemistry Series, Vol. 198, American Chemical Society, Washington, D.C., 1982; Feeney et al., FOOD PROTEINS: IMPROVEMENT THROUGH CHEMICAL AND ENZYMATIC MODIFICATION, Advances in Chemistry Series, Vol. 160, American Chemical Society, Washington, D.C., 1977; and Hermanson, BIOCONJUGATE TECHNIQUES, Academic Press, San Diego, 1996.

Labeling can be conducted and PSTs determined from either the N- or C-terminal end of the protein. About 59-90% of eukaryotic proteins are N-terminal acetylated (see, Creighton, T. E., Proteins: Structures and Molecular Principles (W. H. Freeman, NY, 1984) and are thus refractory to N-terminus labeling. However, the natural N-acetyl group of such proteins can sometimes be used as a label for purposes of this invention, but only where one or more of the amino acids within 4 residues of the N-terminus is ionizable (e.g., is a lysine, arginine, histidine, aspartic acid, or glutamic acid residue) or can be derivatized to be ionizable (e.g., tyrosine, serine, and cysteine residues). Accordingly, strategies to label either the N- or C-termini are provided to afford the greatest degree of sequencing ability for any given protein.

Following the labeling methods described herein, the labeled protein samples are suitable for separation, sequencing and quantitation as described in, for example, co-pending PCT application Ser. No. ______, filed Apr. 19, 2000, entitled “Polypeptide Fingerprinting Methods, Metabolic Profiling, and Bioinformatics Database,” Attorney Docket No. 020444-000600PC.

The methods of the present invention are further illustrated by the examples which follow. These examples are offered to illustrate, but not to limit the claimed invention.

### EXAMPLES

### Example 1

Labeling Proteins with a Fluorescent Tag for MDE and IMLS Analysis of Diseased and Healthy Tissue Samples for the Purpose of Creating a Proteomics Database Profile

In cases where protein concentrations are low enough to require a more sensitive mode of detection, the use of a fluorophore to achieve the desired sensitivity is well documented. Further, in the case of performing labeling reactions, it is common to implement a protection strategy to ensure that only the desired groups are labeled. The labeling of a protein with various agents in an aqueous or mixed aqueous/organic solvent milieu is also known in the art and a wide range of labeling reagents and techniques useful in practicing the present invention are readily available to those of skill in the art. See, for example, Means et al., CHEMICAL MODIFICATION OF PROTEINS, Holden-Day, San Francisco, 1971; Feeney et al., MODIFICATION OF PROTEINS: FOOD, NUTRITIONAL AND PHARMACOLOGICAL ASPECTS, Advances in Chemistry Series, Vol. 198, American Chemical Society, Washington, D.C., 1982; Feeney et al., FOOD PROTEINS: IMPROVEMENT THROUGH CHEMICAL AND ENZYMATIC MODIFICATION, Advances in Chemistry Series, Vol. 160, American Chemical Society, Washington, D.C., 1977; and Hermanson, BIOCONJUGATE TECHNIQUES, Academic Press, San Diego, 1996.

The following example illustrates how a fluorogenic PST is selected and utilized in a typical embodiment of the invention. In addition, the example illustrates the strategic use of protective group chemistry (see Greene and Wuts, Protective Groups in Organic Synthesis, 3rd Ed., Wiley Science (1999)) to ensure selective terminal labeling of the proteins or peptides. Finally, this example illustrates the prudent use of isotopic tags and modifiers to produce unique mass fragments when the material is fragmented and analyzed under IMLS conditions (see co-pending application Ser. No. 09/513,395, filed Feb. 25, 2000).

In this example, tissue samples from healthy and diseased specimens are subjected to typical procedures for raw extraction of proteins. A 100 mg protein sample is dissolved in 1% TCEP at a concentration of 5 mg/mL and denatured for 15 min at 95° C. In a variation, individual proteins or protein fractions are separated from the mixture prior to labeling, such as described in copending application Ser. Nos. 09/513,486 and 09/513,907. The proteins are then subjected to labeling with Quantum Dye™, a commercially available compound (Research Organics, Cleveland, Ohio) Each of the fractionated test and control protein samples are dissolved to a concentration of 0.5-3 mg/mL in 0.05M borate (or carbonate) at a pH of 9-9.5. 0.4 M sodium chloride is added to adjust ionic strength. A suitable volume (5 mL) is placed in a dialysis bag. Approximately 3 mg of the Quantum Dye™ is dissolved into 100 mL borate (carbonate) buffer, pH 9.3. The protein solutions are dialyzed against the dye solutions over a suitable time (1 hr+) at room temperature to conjugate the tag to the proteins. Quantum Dye™, a commercially available compound (Research Organics, Cleveland Ohio), is a macrocyclic chelate of Europium (III) that exhibits unique fluorescent properties (see, Leif, et al., ACS Symposium Series 464, Cell Separation Science and Technology, D. S. Kampala and P. W. Todd Editors, American Chemical Society, Washington, D.C., PP. 41-58 (1991); Vallarino, et al., Proceedings of Advances in Fluorescence Sensing Technology, J. R. Lakowicz and R. B. Thompson, Editors and A. Katzir, Progress in Biomedical Optics Series Editor, SPIE Proceeding Series 1885 pp. 376-385 (1993); and Leif, et al., Proceedings of Biochemical Diagnostic Instrumentation, Progress in Biomedical Optics. Ed., R. F. Bonner, G. E. Cohn, T. M. Laue, and A. V. Priezzhev. SPIE Proceedings Series 2136, pp. 255-262 (1994)) (See FIG. 1) and incorporates the primary features of the invention. When excited by light of the proper energy (360 mn), and in the presence of an enhancing agent, the Eu (III) chelate exhibits an intense narrow-band emission spectrum (typically around 10 nm at one-half the peak height) with a large Stoke's shift. The complex fluoresces strongly with an emission around 620 nm. The characteristically long fluorescent times (>300 microseconds, see, Periasamy, et al., Microscopy and Analysis, March pp. 33-35 (1995) and Seveus, et al., Micro. Res. And Tech. 28, pp. 149-154 (1994)) enable one to conduct short-pulsed excitation of the sample followed by time-delayed signal detection consequently zeroing out background interference from components with relatively shorter fluorescent lifetimes. Fortuitously, the label does not suffer from fluorescent quenching as do many other labels and thus the response curve for standards remains linear at high concentrations. Seveus, et al., ibid. have shown that these compounds are optimal for biological applications due to their increased water solubility and relative inertness to release of the chelated metal. This enables the use of safer reaction conditions and provides an additional feature of a hard positive charge in the label. The phenylisothiocyanate linkage on the Quantum Dye™ is optimum for attachment to amines, and the positive three (+3) hard charge in the center of the chelate brings the tag's large mass of 927.7 amu to a comfortable m/z value of just 309.2333.

In a variation, the labeled proteins are subjected to further separation steps, such as described in copending applications Ser. Nos. 09/513,486 and 09/513,907. Individual labeled proteins may then be sequenced as described in copending application Ser. No. 09/513,395.

The proteins are monitored by fluorescence or ultraviolet detection since the Quantum Dye™ is also a chromophore in addition to being a fluorophore. Each of the fractions are quantitated by fluorescence and UV. The fractions are then partitioned and analyzed for total protein by NanoOrange™ and Sypro™ dyes (Molecular Probes, Inc., see Harvey M. D., et al., Electrophoresis Sep; 19(12):2169-74 (1998)). Knowing the total mass of the proteins analyzed for each sample, the theoretical amount of fluorescent tag required for complete labeling can be estimated. This can be determined in conjunction with a total digest of the fractionated protein to determine the overall amino acid content. The amount of tag actually found within each protein fraction is compared to the theoretical amount expected based on the AA analysis and total mass found by Sypro™ and NanoOrange™ assays. In this way the overall labeling efficiency is determined on a sample-by-sample basis.

In a typical NanoOrange™ or Sypro™ Assay, detection to ng/mL levels are possible. The reagent is non-fluorescent in aqueous solution, but upon interaction with proteins in the presence of detergent, it binds tenaciously to proteins. See Molecular Probes, NanoOrange Protein Quantitation Kit (N-6666), Product Information Brochure MP06666 (Jul. 27, 1999) and Molecular Probes, Sypro Orange and Red Protein Gel Stains, Product Information Brochure MP06650 (Aug. 17, 1999).

In cases where other labels are needed, the choices are many. See, for example, labels provided in FIG. 3 that are commercially available from Molecular Probes and Research Organics. Other linkage chemistries on fluorophores include sulfonyl chloride, NHS esters, alkyl halide, and activated alkene. Choosing other linkage chemistries allows use of other fluorogenic species as tags and increases the chances of having the choices necessary to generate excellent IMLS data with fragments encompassing many amino acid residues. Each of the selections, including the Quantum Dye™ tag used in this example, and many more labels are possible that incorporate the primary features of the invention; i.e. a linking functionality, a charged specie (+ or −), hard or soft charge, and a unique mass signature.

### Example 2

In this example, proteins are labeled and sequenced from the C-terminus. To label proteins on the C-terminal, some protective measures are usually required and desirable (see, Atassi, et al., Eds. Methods in Protein Structure Analysis, Plenum Press, 1995; Boyd, V. L., et al., Sequencing of Proteins from the C-terminus, pp. 109-118; and Dupont, et al., PE Biosystems, Inc. Application Note, http://www.pebio.com). On the basis of data collected from routine AA assays, the proper protective chemistries are selected. It has been demonstrated that cys, lys, ser, thr, asp, and glu side-chain residues can pose challenges when attempting to perform some types of C-terminal labeling or sequencing, and modification of these groups is often necessary, though relatively easy to accomplish (see, for example, Atassi, et al., ibid. and Guga, et al., “C-terminal Sequence Analysis of the Amino Acids with Reactive Side-Chains: Ser, Thr, Cys, Glu, Asp, His, Lys.” Poster presentation at the Seventh Symposium Of the Protein Society (1993)).

These protective chemistries are similar to those used by PE Biosystems in their Procise 494C Sequencer (see, Werner, et al., “A New Simple Preparation Device for Protein/Peptide Sequencing” Poster presentation at the Ninth Symposium of the Protein Society; and Brune, Anal. Biochem. 207:285 (1992)). Cys residues are optionally modified by alkylation with acrylamide to yield stable derivatives. The protein is dissolved into an acrylamide monomer mixture at a 1:10 mole ratio and let sit at room temperature for 2 hours. Lys, Ser, and Thr are optionally modified with phenylthioisocyanate. In a variation, these residues may be modified with a mixture of isotopic and non-isotopic phenylisothiocyanate. The phenylurea derivatives formed at the lysines and carbamates at the Ser and Thr, and an arylcarbamate at the Tyr residues. In a variation of the method, a mixture of isotopically enriched PITC can be used to yield unique mass fragment pairs in the IMLS spectrum. The choice of how much PITC to use will help with the ultimate hydrophobicity when adhering to PVDF membranes or solubility of the derivatized proteins in the subsequent anhydride conversion step. Dissolve the derivatized protein into a mixture 5 grams of PVDF polymer beads and let sit for 1 hour. Filter mixture through 0.45 um filter to isolate beaded material coated with protein. These conversions are easily carried out on PVDF membranes, but can also be conducted in solution phase.

The hydroxyl groups of Ser and Thr are optionally capped using acetic anhydride in the following steps instead of with PITC. In a variation, it is possible to incorporate an isotopically mixed acetic anhydride on the Thr and Ser residues during the capping process to enable easier identification of those residues later during IMLS analysis. Asp and glu residues along with the C-terminus are also reacted with an excess of neat acetic anhydride to form mixed anhydride derivatives. The C-terminal uniquely forms an oxazolone ring that does not form for either of the asp or glu acidic side residues and is stable to nucleophilic attack at pH greater than 8. The mixed anhydrides formed at the asp and glu residues are then selectively modified by the addition of ammonia or a primary amine at high pH and room temperature, such as by the addition of 10% lutidine or pyridine to the acetic anhydride-protein solution. In a variation, all or a substantial fraction of the reactive ammonia may consist of the ¹⁵N isotope, so that native asn residues can be distinguished from asp and glu residues during sequencing. The C-terminal oxazolone is resistant to modification at this conditions.

In a deviation from the method of Werner et al (see infra), the C-terminal oxazolone is then reacted with any desired amine label (as defined supra). This is accomplished by lowering the pH (below 6) by the addition of trifluoroacetic acid so that the C-terminal oxazolone becomes susceptible to nucleophilic attack. In a variation, the same amine label used to modify the asp and glu residues is used to modify the C-terminus at these conditions. In another variation, excess nucleophile is removed prior to lowering the pH and replaced with a different primary amine label for the C-terminus. The C-terminal label preferably consists of a roughly equimolar ratio of stable isotopes of the same compound to facilitate peptide fragment identification during mass spectrometer sequencing.

Following the labeling methods described herein, the labeled protein samples are suitable for separation, sequencing and quantitation as described in, for example, co-pending PCT application Ser. No. ______, filed Apr. 19, 2000, entitled “Polypeptide Fingerprinting Methods, Metabolic Profiling, and Bioinformatics Database,” and having Attorney Docket No. 020444-000600PC.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of labeling a plurality of different proteins in a protein sample, said method comprising contacting said protein sample with a labeling agent comprising a unique ion mass signature component, a quantitative detection component and a reactive functional group to covalently attach a label to at least a portion of said plurality of different proteins.

2. A method in accordance with claim 1, wherein said protein sample comprises at least five different proteins.

3. A method in accordance with claim 1, wherein said protein sample comprises at least ten different proteins.

4. A method in accordance with claim 1, wherein said protein sample comprises at least 50 different proteins.

5. A method in accordance with claim 1, wherein said protein sample comprises at least 100 different proteins.

6. A method in accordance with claim 1, wherein said protein sample is from a biological sample selected from the group consisting of blood plasma, cerebral spinal fluid, cells and tissues.

7. A method in accordance with claim 1, wherein said quantitative detection component is selected from the group consisting of a radioisotope, a fluorescent residue and a chromophore.

8. A method in accordance with claim 1, wherein said reactive functional group is selected from the group consisting of functional groups reactive to primary amines and functional groups reactive to carboxylic acids.

9. A method in accordance with claim 1, wherein said unique ion mass signature component imparts a mass to a protein fragment that does not match a residue mass for any of the 20 natural amino acids.

10. A method in accordance with claim 1, wherein said unique ion mass signature component imparts a mass to a protein fragment of from about 100 amu to about 700 amu.

11. A method in accordance with claim 1, wherein said unique ion mass signature component incorporates stable isotopes in said labeling agent, said stable isotopes selected from the group consisting of ²H, ¹³C, ¹⁵N and ³⁷Cl.

12. A method in accordance with claim 1, wherein said labeling agent is a mixture of labeling agents comprising two different unique ion mass signature components.

13. A method in accordance with claim 1, wherein said detection enhancement component is a group that imparts a positively charged or negatively charged ionic species under fragmentation conditions in a mass spectrometer ionization chamber.

14. A method in accordance with claim 1, wherein said detection enhancement component carries a hard charge.

15. A method in accordance with claim 14, wherein said hard charge is provided by a member selected from the group consisting of quaternary ammonium, quaternary phosphonium and quaternary borate ester groups.

16. A method in accordance with claim 1, wherein said detection enhancement component carries a soft charge.

17. A method in accordance with claim 1, wherein said detection enhancement component is a fluorophore selected from the group consisting of naphthylamines, coumarins, acridines, stilbenes and pyrenes.

18. A method in accordance with claim 1, wherein said detection enhancement component is a fluorophore selected from the group consisting of 1-dimethylaminonaphthyl-5-sulfonate, 1-anilino-8-naphthalene sulfonate, 2-p-toluidinyl-6-naphthalene sulfonate, 3-phenyl-7-isocyanatocoumarin, 9-isothiocyanatoacridine, acridine orange, N-(p-(2-benzoxazolyl)phenyl)maleimide, and benzoxadiazoles.

19. A method in accordance with claim 1, wherein said labeling agent is selected from the group consisting of sulfo-PITC, compounds of FIG. 1, compounds of FIG. 2 and compounds of FIG. 3.
